(19) United States
(12) Patent Application Publication
(10) Pub. No.: US 2015/0053312 A1
(43) Pub. Date: Feb. 26, 2015

(54) Metallic Glass Film for Medical Application

(71) Applicant: Jinn Chu, (US)

(72) Inventors: Jinn Chu, Keelung (TW); Jyh-Wei Lee, Taipei (TW); Ming-Jen Chen, Taipei (TW); Shih-Hsin Chang, New Taipei City (TW); Jason Shian-Ching Jang, Hsinchu (TW); Wen-Chien Huang, Taipei (TW); Pei-Hua Tsai, Tainan (TW); Jian-Wei Chiou, Hsinchu (TW)

(21) Appl. No.: 13/974,116

(22) Filed: Aug. 23, 2013

(51) Int. Cl. A61L 31/08; C22C 45/00; A61L 31/02; C22C 45/10

(52) U.S. Cl. CPC A61L 31/088; C22C 45/10; C22C 45/001; A61L 31/022
USPC 148/403

(57) ABSTRACT

The present invention relates to a metallic glass film for medical application, which is an amorphous thin film metallic glass (TFMG) formed for covering the surface of a substrate (for example, a medical cutting instrument), so as to increase the wear resistance and the sharpness of the substrate, decrease the surface roughness of the substrate, protect the edge of the substrate from curl and chipping crack. In the present invention, the TFMG is a zirconium-based thin film metallic glass constituted by Zr material, Cu material, Al material, and Ta material with the atom percent of 53 at %, 33 at %, 9 at %, 5 at %, respectively. Moreover, the TFMG can also be constituted by Cu material, Zr material, Al material, and Ti material, and the atom percent of the Cu material, the Zr material, the Al material, and the Ti material are 48 at %, 42 at %, 6 at %, 4 at %, respectively.

### BACKGROUND OF THE INVENTION

1. Field of the Invention

The present invention relates to thin film metallic glass technologies, and more particularly to an amorphous thin film metallic glasses used for being a surface coating of medical cutting instruments.

2. Description of the Prior Art

Metals, ceramics and polymer materials are currently widely applied in clothes, foods, shelters, and transportations of people life, wherein the metal materials further perform the highest application. Comparing to polymer or composite materials, metal materials show better fatigue resistance and creep resistance; besides, because people have been studied metal materials for a very long time, they have sufficient experience for utilizing and processing the metal materials to various industrial and commercial products.

There are some currently used metal materials and the applications thereof. For example, TiAlN film is used for increasing the wear resistance of cutleries and Ti-Si film is applied in enhance the high-temperature stability of cutleries. However, the currently used metal materials still cannot meet the requirements of some specific industries due to their crystal structures and limited ductility, for example, medical cutting instruments.

Differing from above-mentioned crystalline metals, amorphous metals (i.e., so-called metallic glass) include the excellent mechanical properties of high strength, high hardness, and well corrosion resistance, therefore the compositions and manufacturing method of the metallic glasses have been became an important issue. Accordingly, in order to increase the sharpness and decrease the surface roughness of medical cutting instruments, the inventor of the present application has made great efforts to make inventive research thereon and eventually provided an amorphous thin film metallic glass used for being a surface coating of the medical cutting instruments.

### SUMMARY OF THE INVENTION

The primary objective of the present invention is to provide a metallic glass film for medical application, which is an amorphous thin film metallic glass (TFMG) formed for covering the surface of a substrate (for example, a medical cutting instrument), so as to increase the wear resistance and the sharpness of the substrate, and decrease the surface roughness of the substrate, and protect the edge of the substrate from curl and chipping crack.

Accordingly, to achieve the primary objective of the present invention, the inventors propose a metallic glass film for medical application, wherein the metallic glass film is an amorphous thin film metallic glass (TFMG) formed for covering the surface of a substrate, so as to increase the sharpness of the substrate, decrease the surface roughness of the substrate, and protect the edge of the substrate from curl and chipping crack.

In above-mentioned metallic glass film for medical application, wherein the amorphous thin film metallic glass (TFMG) is a zirconium-based thin film metallic glass constituted by a zirconium (Zr) material, a copper (Cu) material, an aluminum (Al) material, and a tantalum (Ta) material, and the atom percent of the zirconium material, the copper material, the aluminum material, and the tantalum material are 53 at %, 33 at %, 9 at %, 5 at %, respectively; moreover, the aforesaid zirconium-based thin film metallic glass of Zr₅₃Cu₃₃Al₉Ta₅ has a glass transition temperature (Tg) of 467.9° C. and a crystalline temperature (Tx) of 519.8° C.

Besides the zirconium-based thin film metallic glass of Zr₅₃Cu₃₃Al₉Ta₅, the aforesaid amorphous thin film metallic glass (TFMG) can also be constituted by a copper (Cu) material, a zirconium (Zr) material, an aluminum (Al) material, and titanium (Ti) material, and the atom percent of the copper material, the zirconium material, the aluminum material, and the titanium material are 48 at %, 42 at %, 6 at %, 4 at %, respectively, wherein the copper-based metallic glass of Cu₄₈Zr₄₂Al₆Ti₄ has a glass transition temperature (Tg) of 467.9° C. and a crystalline temperature (Tx) of 519.8° C.

### BRIEF DESCRIPTION OF THE DRAWINGS

The invention as well as a preferred mode of use and advantages thereof will be best understood by referring to the following detailed description of an illustrative embodiment in conjunction with the accompanying drawings, wherein:

FIG. 1 is a schematic cross-sectional side view of a metallic glass film for medical application according to the present invention;

FIG. 2 is an X-ray diffraction (XRD) analysis plot of a zirconium-based thin film metallic glass and a copper-based thin film metallic glass;

FIG. 3 is a DSC (Differential Scanning calorimetry) analysis plot of the zirconium-based thin film metallic glass and the copper-based thin film metallic;

FIG. 4 is an SEM (Scanning Electron Microscopy) image of a bare substrate;

FIG. 5 is an SEM images of a substrate after being covered with the thin film metallic glass;

FIG. 6 is SEM images of a bare substrate and a substrate after being covered with the thin film metallic glass;

FIG. 7 is surface roughness distribution plots of the substrate before and after being covered with the thin film metallic glass; and

FIG. 8 is cross-sectional optical micrographs of the skin surfaces of pigs after incising with dermatomes.

### DETAILED DESCRIPTION OF THE PREFERRED EMBODIMENTS

To more clearly describe a metallic glass film for medical application according to the present invention, embodiments of the present invention will be described in detail with reference to the attached drawings hereinafter.

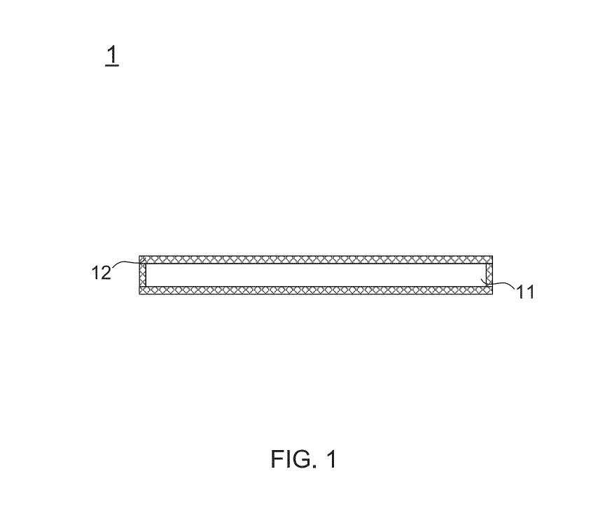

Please refer to FIG. 1, which illustrates a schematic cross-sectional side view of a metallic glass film for medical application according to the present invention. As shown in FIG. 1, the metallic glass film proposed by the present invention is an amorphous thin film metallic glass (TFMG) 12 formed for covering the surface of a substrate 11, so as to increase the wear resistance and the sharpness of the substrate 11, and decrease the surface roughness of the substrate 11. After being coated with the amorphous thin film metallic glass 12, the sharpness of the substrate 11 does be increased with the increment ranging from 20% to 40%; moreover, the surface roughness of the substrate 11 does be decreased with the decrement ranging from 60% to 70%. Herein, it needs to further explain that, the substrate 11 mentioned in the present invention means a kind of medical cutting instruments, for example, a dermatome, which is made of titanium (Ti), titanium alloy, aluminum (Al), aluminum alloy, copper (Cu), copper alloy, iron (Fe), iron alloy, magnesium (Mg), magnesium alloy, nickel, nickel alloy, zirconium (Zr), or zirconium alloy.

Particular, the amorphous thin film metallic glass 12 of the present invention is a zirconium-based thin film metallic glass constituted by a zirconium (Zr) material, a copper (Cu) material, an aluminum (Al) material, and a tantalum (Ta) material, and the atom percent of the zirconium material, the copper material, the aluminum material, and the tantalum material are 53 at %, 33 at %, 9 at %, 5 at %, respectively. Moreover, besides the zirconium-based thin film metallic glass of Zr₅₃Cu₃₃Al₉Ta₅, the amorphous thin film metallic glass 12 of the present invention can also be constituted by a copper-based thin film metallic glass constituted by a copper (Cu) material, a zirconium (Zr) material, an aluminum (Al) material, and titanium (Ti) material, and the atom percent of the copper material, the zirconium material, the aluminum material, and the titanium material are 48 at %, 42 at %, 6 at %, 4 at %, respectively.

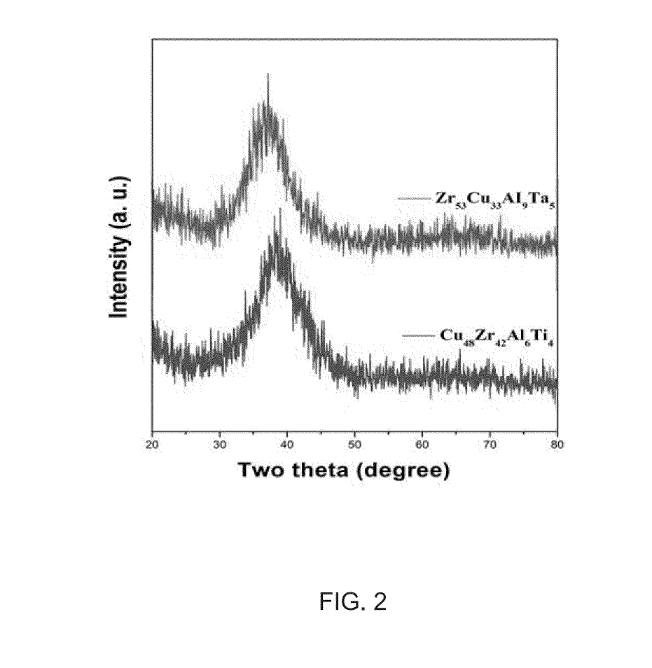

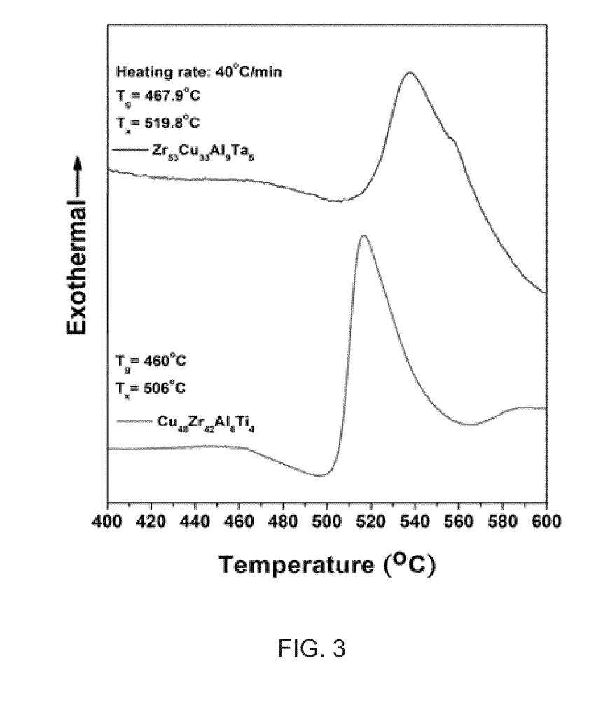

Thus, through above descriptions, the frameworks and the related constitutes of the metallic glass film for medical application of the present invention have been completely introduced and disclosed. Next, in order to prove the practicability of the metallic glass film for medical application, a variety of experimental data will be presented as follows. Please refer to FIG. 2 and FIG. 3, which respectively illustrate an X-ray diffraction (XRD) analysis plot and a DSC (Differential Scanning calorimetry) analysis plot of the zirconium-based thin film metallic glass and the copper-based thin film metallic. As shown in FIG. 2 the XRD plot shows that the TFMG indeed includes an amorphous structure no matter the TFMG is Zr₅₃Cu₃₃Al₉Ta₅ or Cu₄₈Zr₄₂Al₆Ti₄. Moreover, as shown in FIG. 3, the DSC result reveals that the glass transition temperature (Tg) and the crystallization temperature (Tx) of the Zr₅₃Cu₃₃Al₉Ta₅ TFMG are 467.9° C. and 519.8° C., respectively; moreover, the glass transition temperature (Tg) and the crystallization temperature (Tx) of the Cu₄₈Zr₄₂Al₆Ti₄ TFMG are respectively 460° C. and 506° C.

Herein, it needs to further explain that, although above descriptions state that the metallic glass film for medical application proposed by the present invention can be a zirconium-based thin film metallic glass or a copper-based thin film metallic glass, that does not used for limiting the exemplary embodiments of the metallic glass film for medical application. In practical application, the the metallic glass film for medical application can also be yttrium-based metallic glass, vanadium-based metallic glass, titanium-based metallic glass, tantalum-based metallic glass, samarium-based metallic glass, praseodymium-based metallic glass, platinum-based metallic glass, palladium-based metallic glass, nickel-based metallic glass, neodymium-based metallic glass, magnesium-based metallic glass, lanthanum-based metallic glass, hafnium-based metallic glass, iron-based metallic glass, copper-based metallic glass, cobalt-based metallic glass, cerium-based metallic glass, calcium-based metallic glass, gold-based metallic glass, or aluminum-based metallic glass.

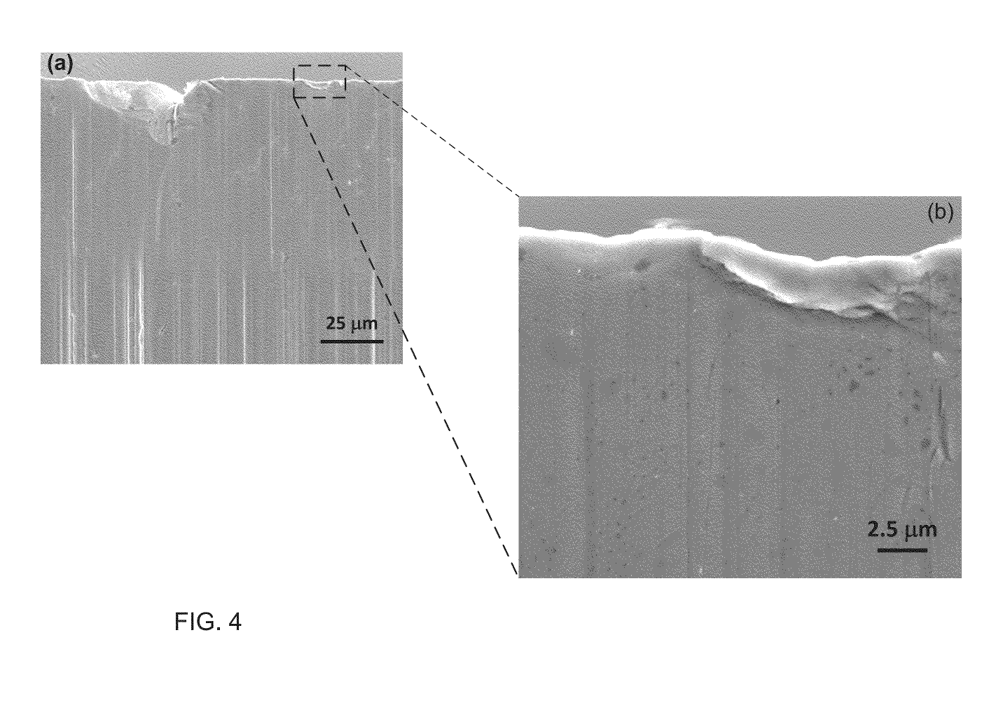

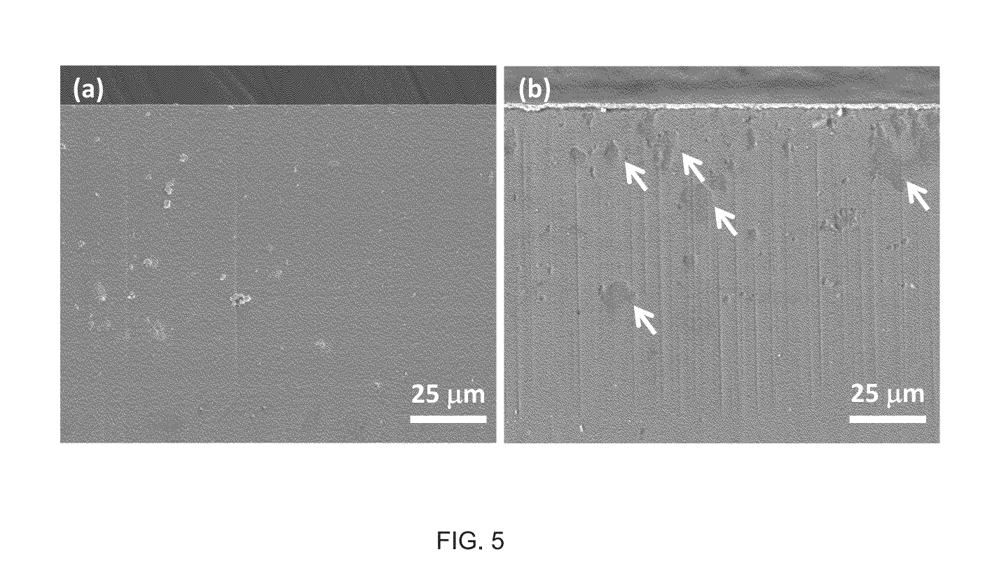

Referring to FIG. 4, there is shown an SEM (Scanning Electron Microscopy) image of the substrate; Moreover, FIG. 5 illustrates an SEM image of a substrate after being covered with the thin film metallic glass. In FIG. 4, images (a) and (b) show a control substrate (i.e., the bare substrate) been treated with a blade sharpness index texting (BSI). After the blade sharpness index texting, as shown by the zoom-in image of images (a), i.e., the images (b), which reveals that there are obvious damages, curls and peelings produced on the surface of the bare substrate. Oppositely, In FIG. 5, images (a) and (b) are respectively the substrate after being covered with the thin film metallic glass of an experiment group been treated a blade sharpness index texting (BSI). After the blade sharpness index texting, as shown by the zoom-in image of images (a), i.e., the images (b), which shows that there are some minor damages and peelings produced on the surface of the substrate of the experiment group. The most important is that, there have no any curls produced on the edge the substrate, but merely a few chipping crack being produced on the surface thereof. Therefore, the SEM images of FIG. 4 and FIG. 5 prove that the amorphous thin film metallic glass 12 of the present invention can indeed increase the wear resistance of the substrate 11 (i.e., the medical cutting instrument), and protect the surface of the substrate 11 from curl and chipping crack.

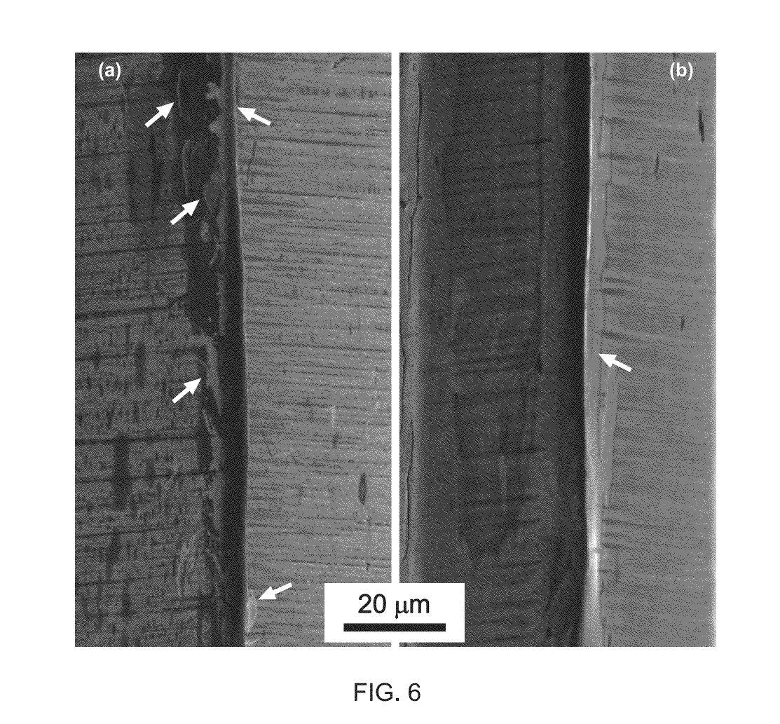

Furthermore, please refer to FIG. 6, which illustrates SEM images of a bare substrate and a substrate after being covered with the thin film metallic glass. In FIG. 6, image (a) shows the control substrate (i.e., the bare substrate) been treated with a blade sharpness index texting (BSI), and the image (a) reveals that there are obvious damages (denoted by the white arrows) produced on the edge of the bare substrate. However, opposite to image (a), image (b) shows the experiment substrate (i.e., the substrate coated with the TFMG) been treated with a blade sharpness index texting (BSI), and the image (b) reveals that there are merely a few damages produced on the edge of the bare substrate. Therefore, the SEM images of FIG. 6 prove that the amorphous thin film metallic glass 12 of the present invention can indeed increase the wear resistance of the substrate 11 (i.e., the medical cutting instrument), and protect the edge of the substrate 11 from suffering damages after being used for cutting. This suggests that the amorphous thin film metallic glass 12 of the present invention has strengthened the edge of the substrate 11, which appears stronger to be split than the bare blade during the BSI test.

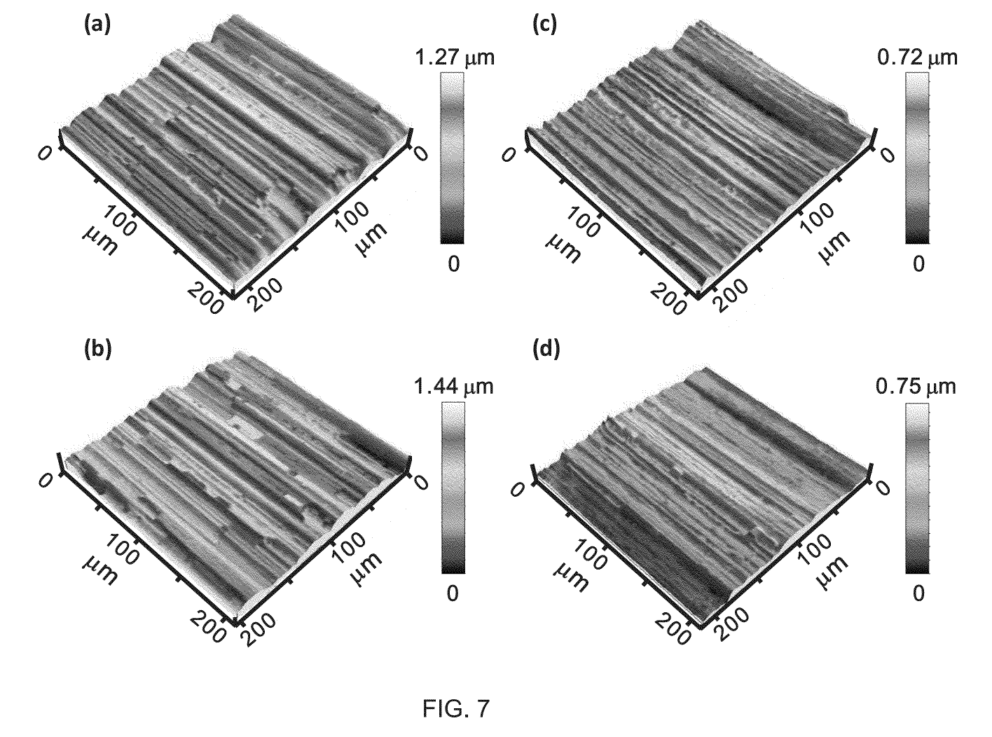

Moreover, please refer to FIG. 7, there are shown surface roughness distribution plots of the substrate before and after being covered with the thin film metallic glass. In FIG. 7, plots (a) and (b) represent the substrate without being covered with the thin film metallic glass, moreover, the substrate in plot (b) has been treated the BSI testing. Oppositely, plots (c) and (d) represent the substrate covered with the thin film metallic glass, and the substrate in plot (d) has been treated the BSI testing. Comparing plot (a) with plot (c), it is able to find that the substrate covered with thin film metallic glass performs a greater surface roughness distribution than the surface roughness distribution of the bare substrate. Moreover, comparing plot (a) with plot (b), the comparison result shows that the surface roughness of the bare substrate is increased after the BSI testing; however, comparing plot (c) with plot (d), the comparison result reveals that the surface roughness of the substrate covered with thin film metallic glass does almost not be increased after the BSI testing. Therefore, the surface roughness distribution plots of FIG. 7 prove that the amorphous thin film metallic glass 12 of the present invention can indeed increase the wear resistance of the substrate 11 (i.e., the medical cutting instrument), and decrease the surface roughness with the decrement ranging from 60% to 70% of the substrate 11.

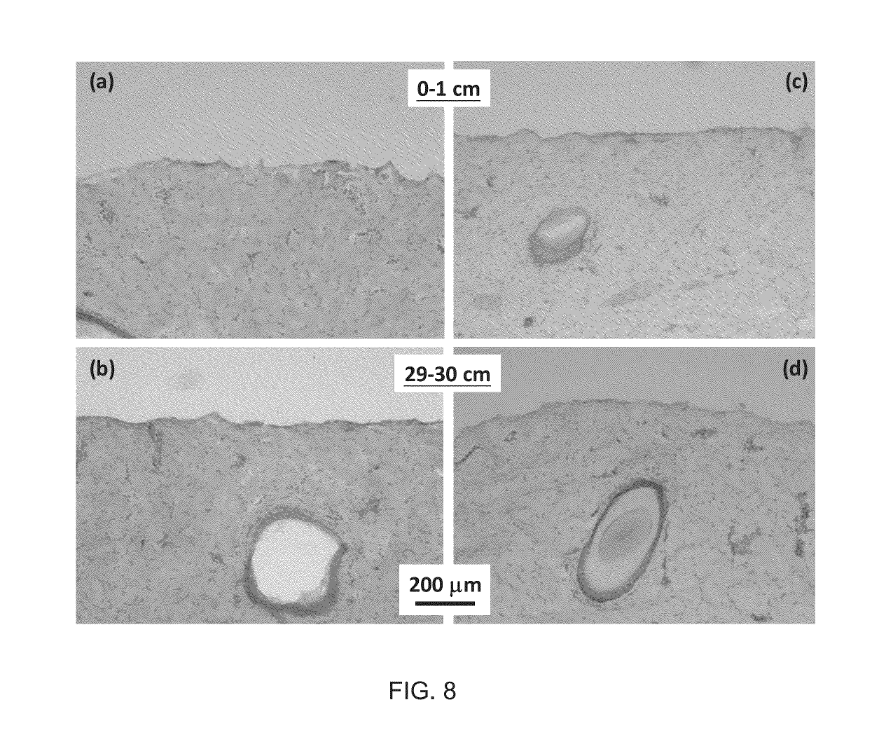

Furthermore, please refer to FIG. 8, there are shown cross-sectional optical micrographs of the skin surfaces of pigs after incising with different dermatomes. In which, FIG. 8(a) and FIG. 8(b) represent the micrographs of two ends of 0-1 cm and 29-30 cm of a dermatome without being covered with the thin film metallic glass. The rough wound skin surface in (a) and (b) is likely attributed to the act of tearing apart the tissue during cutting by the bare dermatome, which is not relatively sharp. Oppositely, FIG. 8(c) and FIG. 8(d) represent the micrographs of two ends of 0-1 cm and 29-30 cm of a dermatome covered with the amorphous thin film metallic glass 12 of the present invention. Comparing plot (a) with plot (c), it is able to find that the dermatome covered with the amorphous thin film metallic glass 12 of the present invention performs a better smooth blade-cutting surface than the bare dermatome. Similarly, comparing plot (c) with plot (d), the comparison result reveals that the dermatome covered with amorphous thin film metallic glass 12 of the present invention glass performs a better smooth blade-cutting surface than the bare dermatome. Therefore, the cross-sectional optical micrographs of FIG. 8 prove that the amorphous thin film metallic glass 12 of the present invention can indeed increase the sharpness of the dermatome with the increment ranging from 20% to 40%. Furthermore, the strengthening by the amorphous thin film metallic glass 12 of the present invention makes the dermatome edge more robust.

Therefore, the above descriptions have been clearly and completely introduced the metallic glass film for medical application (i.e., the amorphous thin film metallic glass (TFMG) 12 of Zr₅₃Cu₃₃Al₉Ta₅ and Cu₄₈Zr₄₂Al₆Ti₄) of the present invention. In summary, the technology feature and the advantage thereof of the present invention is that: the Zr₅₃Cu₃₃Al₉Ta₅ TFMG or the Cu₄₈Zr₄₂Al₆Ti₄ TFMG proposed by the prevent invention can indeed be used for forming and covering a medical cutting instrument (for example, the dermatome), so as to increase the wear resistance and the sharpness of the medical cutting instrument, decrease the surface roughness of the medical cutting instrument, and protect the edge of the medical cutting instrument from curl and chipping crack.

The above description is made on embodiments of the present invention. However, the embodiments are not intended to limit scope of the present invention, and all equivalent implementations or alterations within the spirit of the present invention still fall within the scope of the present invention.

What is claimed is:

1. A metallic glass film for medical application, wherein the metallic glass film is an amorphous thin film metallic glass (TFMG) formed for covering the surface of a substrate, so as to strengthen the edge of substrate, resist the edge of substrate to be split, increase the wear resistance and the sharpness of the substrate, decrease the surface roughness of the substrate, and protect the edge of the substrate from curl and chipping crack.

2. The metallic glass film of claim 1, wherein the material of the substrate is selected from the group consisting of: titanium (Ti), titanium alloy, aluminum (Al), aluminum alloy, copper (Cu), copper alloy, iron (Fe), iron alloy, magnesium (Mg), magnesium alloy, nickel, nickel alloy, zirconium (Zr), and zirconium alloy.

3. The metallic glass film of claim 1, wherein the increment of the sharpness of the substrate is ranged from 20% to 40%, and the decrement of the surface roughness of the substrate is ranged from 60% to 70%.

4. The metallic glass film of claim 1, wherein the amorphous thin film metallic glass (TFMG) is a zirconium-based thin film metallic glass constituted by a zirconium (Zr) material, a copper (Cu) material, an aluminum (Al) material, and a tantalum (Ta) material, and the atom percent of the zirconium material, the copper material, the aluminum material, and the tantalum material are 53 at %, 33 at %, 9 at %, 5 at %, respectively.

5. The metallic glass film of claim 4, wherein the aforesaid zirconium-based thin film metallic glass of Zr₅₃Cu₃₃Al₉Ta₅ has a glass transition temperature (Tg) of 467.9° C. and a crystalline temperature (Tx) of 519.8° C.

6. The metallic glass film of claim 1, wherein the amorphous thin film metallic glass (TFMG) is a copper-based thin film metallic glass constituted by a copper (Cu) material, a zirconium (Zr) material, an aluminum (Al) material, and titanium (Ti) material, and the atom percent of the copper material, the zirconium material, the aluminum material, and the titanium material are 48 at %, 42 at %, 6 at %, 4 at %, respectively.

7. The metallic glass film of claim 6, wherein the aforesaid copper-based metallic glass of Cu₄₈Zr₄₂Al₆Ti₄ has a glass transition temperature (Tg) of 460° C. and a crystalline temperature (Tx) of 506° C.

8. The metallic glass film of claim 1, wherein the amorphous thin film metallic glass (TFMG) is selected from the group consisting of: yttrium-based metallic glass, vanadium-based metallic glass, titanium-based metallic glass, tantalum-based metallic glass, samarium-based metallic glass, praseodymium-based metallic glass, platinum-based metallic glass, palladium-based metallic glass, nickel-based metallic glass, neodymium-based metallic glass, magnesium-based metallic glass, lanthanum-based metallic glass, hafnium-based metallic glass, iron-based metallic glass, copper-based metallic glass, cobalt-based metallic glass, cerium-based metallic glass, calcium-based metallic glass, gold-based metallic glass, and aluminum-based metallic glass.
